(19) United States
(12) Patent Application Publication
(10) Pub. No.: US 2011/0263983 A1
(43) Pub. Date: Oct. 27, 2011

(54) ULTRASOUND IMAGING SYSTEM WITH REMOTE CONTROL AND METOD OF OPERATION THEREOF

(75) Inventor: Michael Peszynski, Newburyport, MA (US)

(73) Assignee: KONINKLIJKE PHILIPS ELECTRONICS N.V., EINDHOVEN (NL)

(21) Appl. No.: 13/141,825

(22) PCT Filed: Dec. 11, 2009
(86) PCT No.: PCT/IB2009/055715

§ 371 Date: Jun. 23, 2011

Related U.S. Application Data
(60) Provisional application No. 61/141,020, filed on Dec. 29, 2008.

(51) Int. Cl. A61B 8/14

(52) U.S. Cl. 600/443; 600/459

(57) ABSTRACT

An ultrasound imaging probe includes a body portion having a rigid section defining at least part of a cavity; a first electromechanical actuator located in the body portion; a second electromechanical actuator located in the body portion; a flexible portion coupled to the body portion, the flexible portion comprising a plurality of articulating elements; a distal part coupled to the flexible portion and defining at least another part of the cavity; and an ultrasonic sensor array situated in the distal part, A controller provides control signals, where a first force transmitting member is coupled to the first electromechanical actuator and at least one of the plurality of articulating elements so as to transfer a force from the first electromechanical actuator to at least one of the articulating elements; and a second force transmitting member is coupled to the second electromechanical actuator and at least another of the plurality of articulating elements so as to transfer a force from the second electromechanical actuator to the other articulating element of the plurality of articulating in response to the control signals from the controller. The controller may be configured to electronically steer a beam from the ultrasonic sensor array in response to a manual manipulation of a joystick by a user to provide volumetric imaging in three dimensions.

The present system relates generally to ultrasound imaging systems for imaging biological tissue, such as a transesophageal echocardiogram (TEE) probe, and, more particularly, to a manual and/or automatic remote controlled transducer which can provide two dimensional (2D) and/or three dimensional (3D) ultrasound image volume, as well as a method of operation thereof.

Typically, during a percutaneous intervention, a surgical instrument such as a catheter must be manually manipulated in order to guide it to a desired location in a patient's body. There are three main methods which are generally used to guide surgical instruments. These are known as an optical imaging method, a fluoroscopic imaging method, and an ultrasound imaging method and will be discussed below.

With regard to the optical imaging method, this method uses a camera such as, for example, a video camera, to capture images of an object at a desired location. These images may then be used to guide the instrument to the desired location in a patient's body. However, as the optical guidance method can only capture images which are in line of site of a lens of the camera, it may be difficult to obtain a detailed image of the surgical implement's location in relation to a patient's body or portions thereof. Accordingly, a surgeon may be incapable of guiding a surgical implement within a user's body with the aid of only an optical guidance method.

With regard to the fluoroscopic imaging method, this method is often used in medical procedures where ultrasound imaging systems are not widely used such as, for example, during cardiac procedures. This method can be used to guide a desired radio dense object such as, a catheter, etc., to a desired location within a patient's body. However, as fluoroscopic imaging does not provide high quality images with good contrast in soft tissue, fluoroscopic imaging may not be suitable for applications in soft tissue regions. Further, as fluoroscopic imaging produces ionizing radiation, it can be hazardous to the patient as well as to persons in contact with, or located within the vicinity of, the patient (e.g., the cardiac interventionalist). Further, medical professionals in the vicinity of the patient may have to wear uncomfortable and bulky lead shielding to shield themselves from potential radiation exposure.

Further, with regard to ultrasound imaging procedures, this method typically uses an ultrasonic probe to obtain digital image data of a desired area of a patient's body. With respect to cardiac imaging, although conventional ultrasonic imaging procedures can be used to obtain images of, for example, the chambers and valves of the heart in spatial and temporal detail sufficient to guide percutaneous cardiac intervention, this method requires a user to manually manipulate a probe in order to obtain desired image information. Accordingly, this method is tedious and time consuming.

Accordingly, there is a need for an automated ultrasound imaging system and method to control endoscopic devices for imaging, manually override the automatic control to obtain desired image and guide the endoscopic devices, and/or generate desired images and information in a percutaneous intervention, such as a percutaneous cardiac intervention.

Further, there is a need for an automated and/or manual control to obtain imaging information and method for an imaging TEE probe guided to a desired image for a percutaneous (e.g., cardiac) intervention.

One object of the present systems, methods, apparatus and devices is to overcome the disadvantages of conventional systems and devices. According to one illustrative embodiment, an ultrasound imaging probe includes a body portion having a rigid section defining at least part of a cavity; a first electromechanical actuator located in the body portion; a second electromechanical actuator located in the body portion; a flexible portion coupled to the body portion, the flexible portion comprising a plurality of articulating elements; a distal part coupled to the flexible portion and defining at least another part of the cavity; and an ultrasonic sensor array situated in the distal part, A controller provides control signals, where a first force transmitting member is coupled to the first electromechanical actuator and at least one of the plurality of articulating elements so as to transfer a force from the first electromechanical actuator to at least one of the articulating elements; and a second force transmitting member is coupled to the second electromechanical actuator and at least another of the plurality of articulating elements so as to transfer a force from the second electromechanical actuator to the other articulating element of the plurality of articulating in response to the control signals from the controller. The controller may be configured to electronically steer a beam from the ultrasonic sensor array in response to a manual manipulation of a joystick by a user to provide volumetric imaging in three dimensions.

The present invention may be introduced into a person's anatomy via, for example, a natural orifice or by percutaneous or surgical access to a lumen, vessel, or body cavity. It should be understood that, although the present system and method will be described in connection with percutaneous cardiac intervention of a person, the percutaneous or surgical intervention and access may be to any percutaneous intervention of any biological being, such as animals, or to non-biological objects such as to probe devices (e.g., electronic devices, inanimate objects, etc.) or structures (e.g., buildings, caves, etc.) through small openings. Further, the present system is also applicable to other forms of doppler effect sonography. Further, although embodiments are described related transesophageal echocardiogram (TEE) probe, the present systems, devices and methods are equally applicable to any endoscopic device for imaging, inserted through any orifice, such as, transnasal, transvaginal, transrectal, endco-cavity probes, etc.

Further areas of applicability of the present devices and systems and methods will become apparent from the detailed description provided hereinafter. It should be understood that the detailed description and specific examples, while indicating exemplary embodiments of the systems and methods, are intended for purposes of illustration only and are not intended to limit the scope of the invention.

These and other features, aspects, and advantages of the apparatus, systems and methods of the present invention will become better understood from the following description, appended claims, and accompanying drawing where:

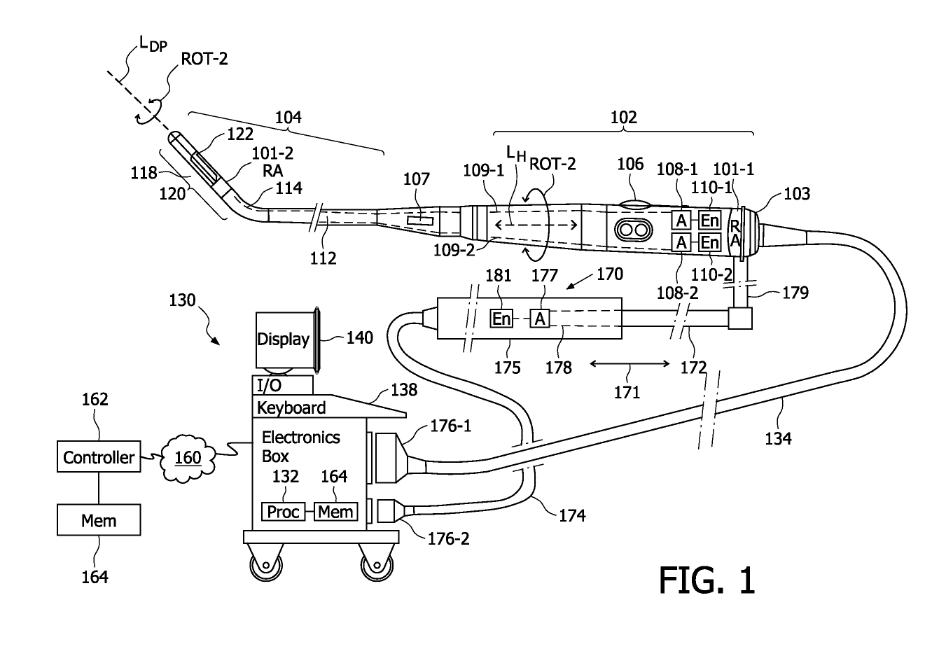

FIG. 1 is an illustration of an ultrasound system for imaging internal tissue according to an embodiment of the present system;

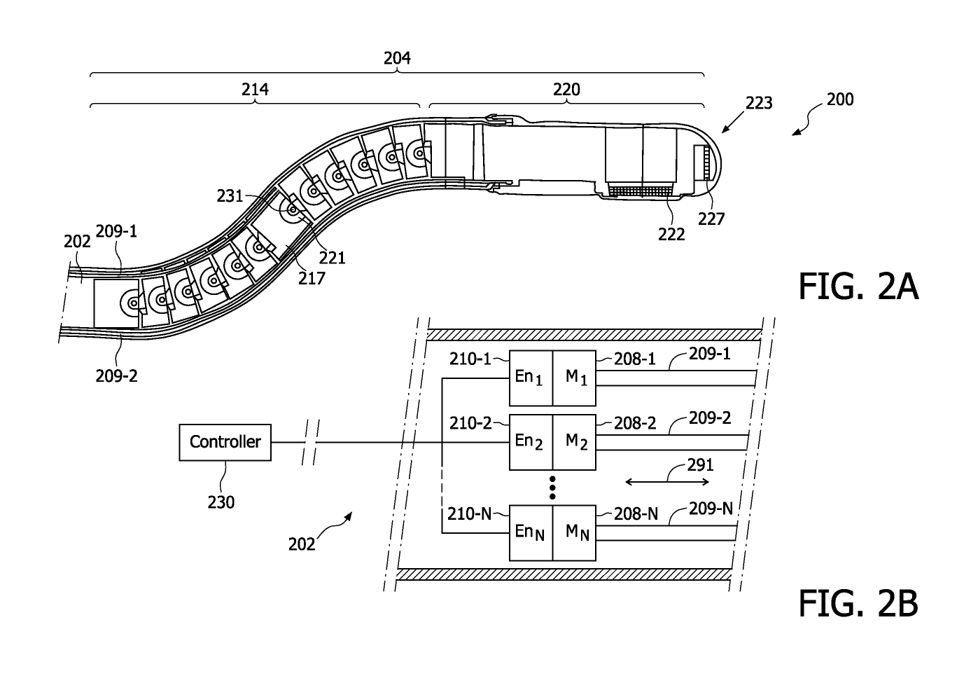

FIGS. 2A-2B show a partial side view illustration of an ultrasound imaging system according to the present system;

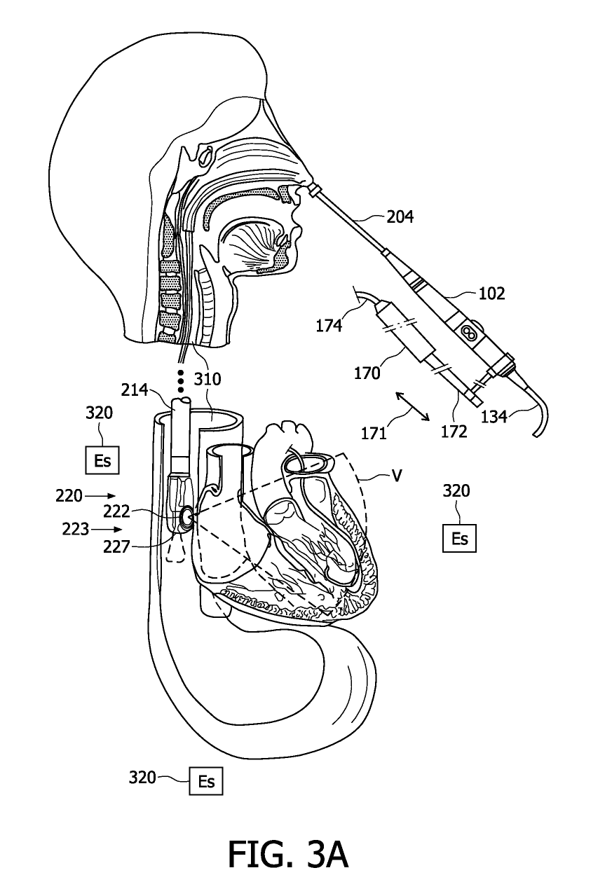

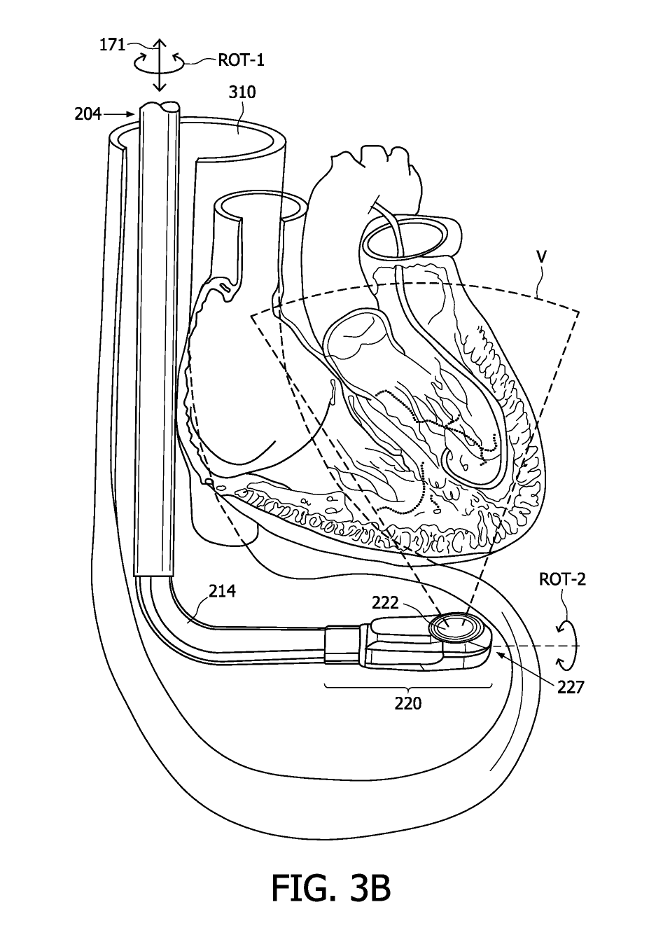

FIG. 3A is an illustration of an endoscopic device for imaging shown in FIG. 2 inserted in a body;

FIG. 3B is an illustration of the imaging endoscopic device shown in FIG. 3A in a bent position within a body;

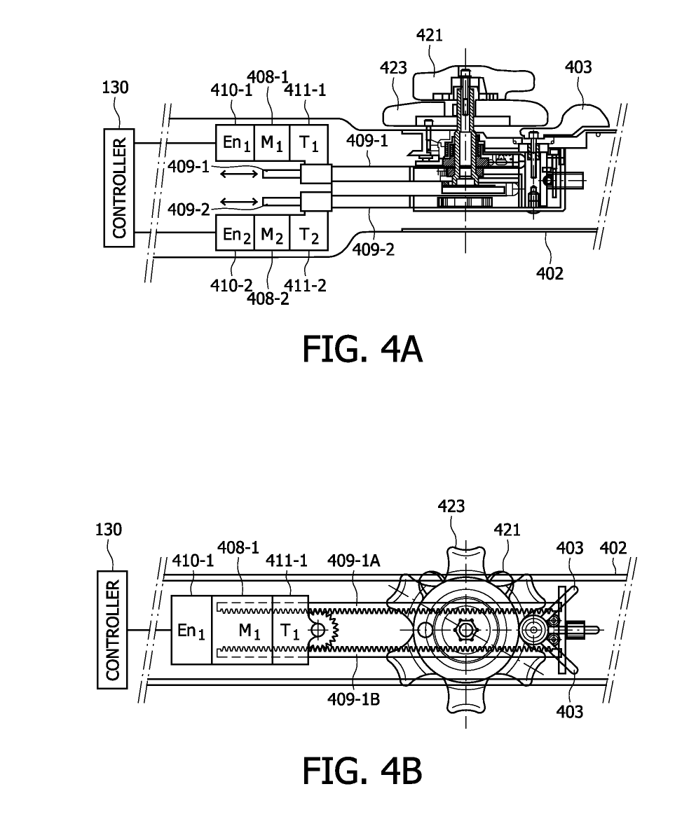

FIG. 4A is a side view illustration of a handle including manual control knobs according to an embodiment of the present invention;

FIG. 4B is a top view illustration of the handle shown in FIG. 4A; and

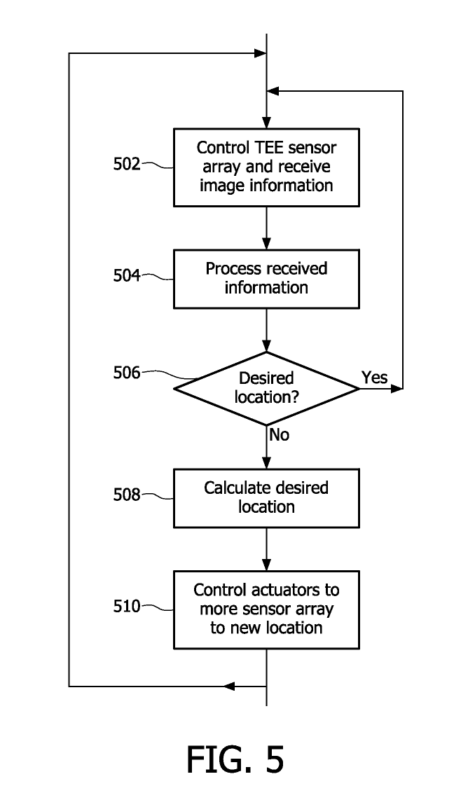

FIG. 5 is a flow chart illustrating a process according to the present system.

The following description of certain exemplary embodiments is merely exemplary in nature and is in no way intended to limit the invention, its applications, or uses. In the following detailed description of embodiments of the present systems and methods, reference is made to the accompanying drawings which form a part hereof, and in which are shown by way of illustration specific embodiments in which the described systems and methods may be practiced. These embodiments are described in sufficient detail to enable those skilled in the art to practice the presently disclosed systems and methods, and it is to be understood that other embodiments may be utilized and that structural and logical changes may be made without departing from the spirit and scope of the present system.

The following detailed description is therefore not to be taken in a limiting sense, and the scope of the present system is defined only by the appended claims. The leading digit(s) of the reference numbers in the figures herein typically correspond to the figure number, with the exception that identical components which appear in multiple figures are identified by the same reference numbers. Moreover, for the purpose of clarity, detailed descriptions of certain features will not be discussed when they would be apparent to those with skill in the art so as not to obscure the description of the present system.

Various imaging systems, probes and controls are known, such as those disclosed in the following U.S. patents or U.S. patent application Publications which are all incorporated herein by reference:

1. U.S. Pat. No. 5,853,368 entitled “Ultrasound Imaging Catheter Having an Independently-Controllable Treatment Structure” issued to Solomon et al. on Dec. 29, 1998;

2. U.S. Pat. No. 6,126,602 entitled “Phased Array Acoustic Systems with Intra-Group Processors” issued to Savord et al. on Oct. 3, 2000;

3. U.S. Pat. No. 6,572,547 B2 entitled “Tranesophageal and Transnasal, Transesophageal Ultrasound Imaging Systems” issued to Miller et al. on Jun. 3, 2003;

4. U.S. Pat. No. 6,592,520 B1 entitled “Ultravascular Ultrasound Imaging Apparatus and Method” issued to Peszynski et al. on Jul. 15, 2003;

5. U.S. Pat. No. 6,679,849 B2 entitled “Ultrasound TEE Probe with Two Dimensional Array Transducer” issued to Miller et al. on Jan. 20, 2004;

6. U.S. Pat. No. 6,776,758 B2 entitled “RFI-Protected Ultrasound Probe” issued to Peszynski et al. on Aug. 17, 2004;

7. US 2004/0073118 A1 entitled “RFI-Protected Ultrasound Probe” to Peszynski et al. and Published on Apr. 15, 2004; and

8. US 2006/0167343 A1 entitled “Control Mechanism for an Endoscope” to Peszynski et al. and Published on Jul. 27, 2006.

An ultrasound system for imaging internal tissue according to an embodiment of the present system is shown in FIG. 1. An ultrasound imaging system 100 may include one or more of a body portion or handle 102, a catheter or an endoscopic device for imaging 104, a telescoping member 170, a control unit 130, a network 160, a control interface 162, one or more memories 164, and one or more control cables 134, 174 which are connectable to the control unit 130 via connectors 176-1, 176-2, respectively.

One embodiment of the endoscopic device for imaging 104 is a transesophageal echocardiogram (TEE) probe for insertion into an esophagus, where such a TEE probe is used for describing the present devices, systems and methods. However, it should be understood that any other type of probe may be used in any desired surgical and imaging applications such as for insertion into any bodily orifice, such as the throat, nose, rectum, etc. Further, the inventive endoscopic devices for imaging according to the present devices, systems and methods may be used alone or in conjunction with surgical instrument for performing desired surgery, such as removal or destruction of undesired growth or tissue, etc. The inventive endoscopic devices may be used for non-invasive or minimally invasive procedures for therapeutic and imaging purposes, and may be self-guided, such as automatically and/or manually e.g., using a joystick, or guided using any conventional guiding devices.

The handle 102 may include one or more internal cavities, one or more actuators (A) 108-1, 108-2, one or more rotational actuators 101-1 and 101-2, a manual override 106, and a support 103. The handle 102 may be coupled to the imaging endoscopic device 104. The one or more actuators (A) 108-1, 108-2, as well as the one or more rotational actuators (RA) 101-1, 101-2, may include any device suitable for generating and transmitting a force such as, for example, motors, solenoids, etc. The one or more rotational actuators 101-1 and 101-2 may rotate parts of the ultrasound imaging system relative about a desired axis. For example, a handle rotational actuator 101-1 may be used to rotate ROT-1 the handle portion 102 about its longitudinal axis L_{H}, or some other axis, as desired. Likewise, a distal rotational actuator 101-2 may be used to rotate ROT-2 a distal part 120 of the imaging endoscopic device 104 about its longitudinal axis L_{DP}, or some other axis, as desired. Further, the handle rotational actuator 101-1 may be used to rotate the handle relative to a support 179 and the distal rotational actuator 101-2 may be used to rotate the distal part 120 relative to a flexible region 114.

The one or more actuators 108-1, 108-2 may receive control signals from the control unit 130 via the cable 134 and may output a corresponding force and/or motion. The force and/or motion output by the one or more actuators 108-1, 108-2 may to be coupled to force transmitting members 109-1, 109-2, respectively (e.g. wires shown as dashed lines), using any suitable coupling. Each of the actuators (e.g., 101-1, 101-2, 108-1, 108-2, and/or actuator 177 of the telescoping member 170) may include a transmission, gears and the like which may multiply or lessen an input force and/or displacement, and output this increased/decreased output rotational speed and/or torque output from, for example, a drum (e.g., for driving a cable, etc.).

Displacement encoders (En) 110-1, 110-2 may transmit position information relating to positions of the actuators 108-1, 108-2, 101-1, 101-2, and/or the force transmitting members 109-1, 109-2, to the control unit 130. The encoders (En) 110-1, 110-2 may also receive corresponding information from the control unit 130. Further, detectors located at the distal part 120 to detect and provide feedback may be provided as desired, such as force detectors to provide tactile feedback, such as by monitoring and limiting the current to the motors, actuators, solenoids, etc. Further, a force gauge may be provided to monitor the tension on the control cables, for example.

The one or more force transmitting members 109-1, 109-2 may include, for example, cables, wires, linkages, racks (e.g., geared racks), and/or combinations thereof. For example, in one embodiment, the one or more of the force transmitting members 109-1, 109-2, may include a geared rack. This geared rack may be coupled to a pinion which is coupled to an output shaft of an electrical motor of a corresponding actuator 108-1, 108-2. Accordingly, the force transmitting members 109-1, 109-2, may receive a force and/or displacement from, for example, the pinion. The force transmitting members 109-1, 109-2 may include corresponding cables which are coupled to the racks.

The endoscopic device for imaging 104 may include one or more cavities which extend along a longitudinal length thereof, a distal part 120, an elongated part 112, and the flexible region 114.

The distal part 120 may include a rigid region 118 and one or more TEE sensor arrays 122. The TEE sensor array 122 may include one or more transducer arrays each of which may include a plurality of ultrasonic elements. The ultrasonic elements may be disposed linearly on an imaging core, for example, and may be coupled to a flex circuit 107. The flex circuit 107 may couple the ultrasonic elements of the one or more transducer arrays and/or other devices within the distal part 120 to the control unit 130 via the cable 134. A TEE sensor control mechanism may be used to control the orientation and/or position of the one or more transducer arrays within the distal part 120. In one embodiment, the TEE sensor control mechanism may include, for example, one or more cables which are coupled to corresponding ones of the one or more transducer arrays so as to control the orientation (which may include roll, pitch, and/or yaw) and/or position of one or more of the transducer arrays. The one or more cables may be coupled to corresponding actuators which may be controlled by the control unit 130 and/or a user.

The TEE sensor arrays 122 may include any suitable ultrasonic sensor arrays such as, for example, a phased array, linear array, curvi-linear array and/or matrix sensor array. Such sensors are disclosed in, for example, U.S. Pat. No. 6,126,602. Other sensor arrays may include matrix array TEE probes, etc. As sensor arrays are known in the art, for the sake of clarity, a further discussion thereof will not be given, and provide to electronic beam steering to view desired images at different location and angles in lieu of a mechanical rotator to rotate the image sensors. Of course if desired, both mechanical and electronic steering of the image beam(s) may be combined as desired.

The elongated part 112 may be substantially rigid and may include a cavity which extends along a longitudinal length thereof. The elongated part 112 may be situated between the distal part 120 and the handle 102 and may couple these two units together.

The flexible region 114 may couple the distal part 120 to the elongated part 112. The flexible region 114 may include a plurality of articulated elements (e.g. similar to articulated elements 217 described below in connection with FIG. 2) which are configured and arranged to provide for the articulation of the rigid region 118 relative to the elongated part 112. The articulated elements (also known as endoscopic flexible links) may be coupled to corresponding actuators 108-1, 108-2 via, for example, corresponding force transmitting members 109-1, 109-2.

A positioning device such as the telescoping member 170 may be included to position the handle in a desired position and/or orientation. It should be understood that although FIG. 1 shows the telescoping member 170 along and connected to the handle via the support 179, the telescoping member 170 may also be in-line or along the longitudinal axis L_{H} of the handle 102 to effectuate movement of the handle 102 along the longitudinal axis L_{H}. Of course, any other positioning device may be used, such as ones with various linkages to provide additional degrees of freedom to effectuate movement and/or rotation of the handle 102 in various directions.

The telescoping member 170 may include a body portion 175 and a telescopic portion 172 which can telescope relative to the body portion 175. The telescopic portion 172 may be coupled to the support 103 of the handle 102 via the support 179. The telescopic member 170 may include one or more actuators 177 which may transmit a force/displacement to the telescopic portion 172, e.g., through wires, piston, or other force transmitting elements 178, so as to cause the telescopic portion 172 to respond accordingly. For example, in one embodiment, the telescopic portion 172 may telescope in a direction which is parallel to the longitudinal axis of the body portion 175 as indicated by arrow 171 in response to a force/displacement from the one or more actuators 177. The telescoping member 170 may include one or more encoders 181 which may generate position information corresponding to a position and/or orientation of the telescopic portion 172 relative to the body portion 175. This position information may be transmitted to, for example, to the control unit 130 via, for example, the control cable 174. Although a single support 179 is shown, it is also envisioned that other supports may be included to support the handle 102. It is also envisioned that the positioning device may be integrated into the handle 102 or may be placed in a parallel or serial configuration with the handle 102.

It is also envisioned that the telescoping member may include two or more arms which are hingedly attached to each other. In yet other embodiments, it is envisioned that the telescoping member include a parallel arm arrangement.

The control unit 130 may include one or more of a display 140, an input/output device 138 such as a joystick, keyboard, mouse, speakers etc., a control unit processor or controller (PROC) 132, a memory (MEM) 164, etc. The control unit 130 and/or processor 132 may control the overall operation of the ultrasound imaging system 100. The control unit 130 may communicate with an external controller 162 via a network 160 which may include a wired and/or wireless network such as, for example, a local area network (LAN), a wide area network (WAN), the Internet, an intranet, etc. Accordingly, the control unit 130 may communicate with further external devices, such as, for example, a remote memory, a remote external control unit, etc. The control unit 130 may control the ultrasound imaging system 100 as set forth in U.S. Pat. No. 6,679,849 (hereinafter the '849 patent) and U.S. Pat. No. 6,592,520 (hereinafter the '520 patent). Accordingly, the TEE sensor array 122 can be controlled to obtain desired information which can be processed and/or displayed as set forth in the '849 and '520 patents. Any suitable transmission scheme may be used to transmit information between different devices of the ultrasound imaging system 100. However, it may be preferred that a proprietary and/or encoded transmission schemes may be used to provide security for information transmitted via the network 160.

The input/output device 138 may include any suitable device or devices which can transmit information to a user and/or receive a user's input. For example, the input/output device 138 may include one or more of a joystick, keyboard (KB) and a pointing device such as, for example, a mouse, a trackball, a touchpad, a capacitive positioning pad, a laser pointer, a touch-screen, etc. The processor may be configured to automatically control the TEE probe to provide desired images, such as in response to preprogrammed or predetermined instructions stored in the memory and executed by the processor, which may be modified or response to various input, such as input from positional and/or force sensors located at the distal end 120, and/or in response to user input. That is, the automatic control to capture desired images may be overridden by manual control by the user based on visual feedback provide by the images captured by the TEE probe, using the joystick, for example, to provide control in x, y and z directions for example. Of course, the opposite may also be provided, where the automatic mode may override the manual mode based on sensor feedback, such as based on force feedback that may indicate a dangerous scenario where any additional manual force is automatically limited to prevent damage, based on predetermine force thresholds, for example, when compared with the actual measured force. Thus, when the actual measure force reaches the threshold, than any further force is not applied. However, after warning or indication which may be acknowledged by the user, the user may be provided with the option to continue, e.g., to continue manual control of the TEE probe despite elevated force feedback signals, for example.

Upon release of the manual control, or activation of the automatic mode by the user, such as by activating a key on the keyboard, the system reverts back to the automatic mode. Thus, a combination of automatic and manual mode is provided where desired images may be captured and displayed on the screen 140, where the user may override the automatic mode anytime. Of course, the system may respond to various types of user inputs, in addition to using the joystick and/or activating buttons, such via voice control where a voice recognition unit recognizes the user's spoken words and translates them to command to control and position the TEE probe to obtain desired images.

The display 140 may include any suitable display for displaying information to a user and may include, for example, a liquid crystal displays (LCD), a touch-screen display, etc. Further, one or more of the displays may be mounted adjacent to another display and/or at a remote location (e.g., in another room, building, city, etc.).

FIG. 2A is a partial side view illustration of an ultrasound imaging system according to the present system. The ultrasound imaging system 200 includes one or more of a handle 202, and a catheter or an endoscopic device for imaging 204. The imaging endoscopic device 204 may include one or more of a flexible region 214 and a distal part 220. The distal part 220 may include one or more ultrasonic sensor arrays such as, for example, a TEE sensor arrays 222, 227, located at different locations and pointed at different directions. For example one TEE sensor array 222 may be located at a lower surface of the distal part 220 pointing down as shown in FIG. 2, where another one TEE sensor array 227 may be located at a front surface of the distal part 220 pointing forward. Of course, additional TEE sensor arrays may also be provided as desired, such as an array pointing up and located at the upper surface of the distal part 220.

Each of the one or more ultrasonic sensor arrays may include one or more sub-arrays. The ultrasound imaging system 200 may include a control unit for positioning and pointing one or more of the TEE sensor arrays in a desired position such that they may obtain image information related to a desired image volume. The distal part 220 may be coupled to the flexible region 214.

The flexible region 214 may include any suitable articulation system such as, for example, a plurality of articulating elements 217, similar to those described in U.S. Pat. No. 6,572,547. The articulating elements 217 may be coupled to each other via one or more joints 231. End parts 221 may be coupled to adjacent articulating elements 217 via corresponding joints 231. One of the end parts 221 may be coupled to an adjacent distal part 220 while the other of the end parts 221 may be coupled to the handle 202. The joints 231 may include hinges or may be formed from a unitary member which can be deflected when subject to a given force. Further, when the joints are formed from a unitary member, the articulating elements may be integrally formed with the joints and/or each other.

As shown in FIG. 2A, the handle 202 may include one or more actuators 208-1 to 208-N and corresponding encoders (En) 210-1 to 210-N. The one or more actuators 208-1 to 208-N may include any suitable force generating mechanism such as motors (M), solenoids, etc. The one or more actuators 208-1 to 208-N may be coupled to corresponding force transmitting members 209-1 to 209-N. The force transmitting members 209-1 to 209-N may be displaced in a linear direction as indicated by arrow 291. The one or more actuators 208-1 to 208-N may receive control signals from, for example, the control unit 130 (FIG. 1) and respond accordingly.

A user interface may be included on, for example, the handle 202 to receive a user's input. Information related to this user input, or control signals from a controller 230, such as from remote controller or the control unit 130, may then be transmitted to the control unit including the processor 132 (of the control unit 130 shown in FIG. 1), for example, which may output one or more signals to control corresponding ones of the one or more actuators (e.g., 101-1, 101-2, 208-1 to 208-N, and/or 177). It is also envisioned that the control signals may be transmitted directly from the user interface to one or more corresponding actuators without being processed by the processor 132. The user interface may include mechanical and/or an electrical interface.

The force transmitting members 209-1 to 209-N may couple a force and/or displacement between the one or more actuators 208-1 to 208-N and corresponding articulating elements 217. The articulating elements 217 may be deflected in one or more planes. Accordingly, the flexible region 214 may be articulated so that it can assume any desired configuration such as, for example, a straight, a “J,” an “S,” and a “Z,” configuration, as desired. Further, the flexible region 214 may also be configured in an out-of-plane configuration. Thus, by precisely controlling the deflection of the force transmitting members 209-1 to 209-N, the articulating elements 217 can be positioned so as to provide articulation of flexible region 214. Accordingly, the imaging endoscopic device 204 may be easily advanced when it is located in a subject mass such as, for example, a gastrointestinal tract, and/or vascular system. Further, the position and/or orientation of TEE sensor array 222, 227 may be easily controlled relative to the subject mass thus enabling the subject mass to be easily examined. As these configurations are known in the art, for the sake of clarity a description thereof will not be given.

An illustration of the imaging endoscopic device 204 shown in FIG. 2A inserted in a body is shown in FIG. 3A. The endoscopic device for imaging 204 is inserted in desired pathway such as, for example, an esophagus 310 (e.g., through the nose as shown in FIG. 3A or through the mouth) and location information from, for example, the TEE sensor array 222 and/or external/internal location devices is transmitted to a control unit, such as the control unit 130 shown in FIG. 1. The location information may be processed and corresponding control signals may be transmitted to one or more of the one or more rotational actuators 101-1 to 101-2, actuator 177 (FIG. 1), and/or 208-1 to 208-N (FIG. 2B). A corresponding force and/or displacement may then be transmitted from the driven actuators. For example, the control unit 130, when instructions loaded in the memory 164 are executed by the processor 132 and/or in response to user input, may control the rotational actuator 101-1 to rotate the rigid region 118. Thus, control signals may be provided by the control unit 130 and/or the processor 132, in response to feedback information, such as location information of the imaging endoscopic device 204 (e.g., its distal portion 220) and/or user input. It should be understood that reference to the control unit 130 is equally applicable to the processor 132. Similarly, the control unit 130 may control one or more of the actuators 108-1 to 108-2 (FIG. 1) to deflect corresponding ones of the articulating elements 217 such that the flexible region 214 (FIG. 2) can be articulated and assume a desired configuration. For example, the flexible region 214 may assume an “L” configuration within a body as shown in FIG. 3B.

The location information may include information related to a location of the imaging endoscopic device 204 relative to one or more external sensors (ES) 320, where three external sensors 320 are shown in FIG. 3A and may use triangulation to determine the imaging endoscopic device location using positional feedback to provide volumetric ultrasound scanning to provide 3D (and/or 2D) images, for example, as described in U.S. Pat. No. 7,270,634 to Scampini et al. entitled “Guidance of Invasive Medical Devices by High Resolution Three Dimensional Ultrasonic Imaging,” which is incorporate herein by reference in its entirety.

Further, the location information may include information received from, for example, a user, and/or the TEE sensor array 222. For example, location information received from the TEE sensor array 222 may include image information obtained in a subject mass. This information may be processed by the control unit 130 and points of interest may be determined. Upon determining a location of the TEE sensor array 222 relative to the point of interest, the control unit 130 may control appropriate actuators so as to cause, for example, the flexible region 214 and/or the telescopic member 172 to remain in a desired position or deflect so as to guide the TEE sensor array 222 to another position. Accordingly, new location information may be obtained from, for example, the TEE sensor array 222 in this new position.

The TEE sensor array 222 may include a plurality of TEE sensor arrays so as to obtain image information corresponding with desired regions about the distal part 220 of the imaging endoscopic device 204. For example, the distal part 220 may include three TEE sensor arrays situated about 120 degrees apart from each other. Further, a TEE sensor array may be mounted at an end 223 of the distal part 220 so as to obtain image information corresponding with the end 223 of the distal part 220. This image information may be included in the location information.

It is also envisioned that the ultrasound imaging system may include image recognition software/hardware so as to render an image and/or determine the location of portions of the imaging endoscopic device 204 such as, for example, the TEE sensor array 222, and/or the location of other desired items, such as catheters with surgical instruments and/or regions of interests, such as body parts to detect tumors or abnormalities, for example. Accordingly, the control unit 130 may use location information and/or information related to a user's input to guide, for example, the TEE sensor array 222 into a desired position and/or orientation. As described, instead of mechanical rotation to change the orientation, electronic beam steering maybe used under the control of the processor 132.

For example, the control unit 130 may control one or more of the actuators 108-1, 108-2, 177, 208-1 to 208-N, and/or the rotational actuators 101-1, 101-2 so as to orient the TEE sensor array 222 in a desired position relative to a tissue volume of interest. The control unit 130 may then engage a braking mechanism to hold the TEE sensor array 222 in a desired position. The control unit 130 may then control the sensor array 222 to obtain image information (e.g., echo information) corresponding to a desired tissue volume. This image information may then be transmitted to the control unit 130 for processing. The external sensors (ES) 320 may transmit information relating to positions of one or more parts of the ultrasound imaging system to the control unit 130. This information may then be processed and used by the control unit 130 to determine positions of one more parts of the ultrasound imaging system. The ultrasound imaging system may also include conventional control knobs as is known in the art and disclosed in, for example, U.S. Patent Publication No. 2006/0167343.

A side view illustration of a handle including manual control knobs according to an embodiment of the present invention is shown in FIG. 4A. An ultrasound imaging system 400 may include one or more of a handle 402, control knobs 421, 423, force transmitting members 409-1, 409-2, and actuators, e.g., motors (M) 408-1, 408-2.

The control knobs 421, 423 may be coupled to the force transmitting members 409-2, 409-1 respectively. Each of the force transmitting members 409-1, 409-2 may include one or more racks. For example, force transmitting member 409-2 may include one or more racks 409-1A, 409-1B that may include teeth for engagement with a gear wheel (e.g., see, FIG. 4B). Likewise, force transmitting member 409-1 may include one or more racks 409-1A and 409-2B. Each of the actuators (M) 408-1, 408-2 may be coupled to force transmitting members (TM) 409-1, 409-2, respectively via corresponding transmissions (T1) 411-1 and (T2) 411-2. The transmissions (T1, T2) 411-1, 411-2 may include an output gear such as pinion. Accordingly, the output gear may be coupled to a corresponding rack via one or more corresponding output gears.

Encoders 410-1, 410-2 may be coupled to corresponding actuators (M1, M2) 408-1, 408-2 and may provide position/location information to the control unit 130. The Encoders 410-1, 410-2 also receive control signals form the control unit 130 for controlling the actuators (M1, M2) 408-1, 408-2. A clutch assembly may be used to couple/decouple forces between the actuators and the control knobs. The clutch assembly may be controlled by a user and/or the control unit 130. An optional locking member or brake mechanism 403 may lock one or more of the force transmitting members 409-1, 409-2 in a desired position. The locking member 403 may be controlled by the user and/or the control unit 130. One or more brake mechanisms may be included to restrict one or more of the actuators and/or force transmitting members from moving from a predetermined position, such as by applying a constant voltage to that the actuators do not move, and/or providing an external or addition braking or locking device, applying closed loop feedback to control the motor and/or actuators and hold them in a desired position. The one or more brake mechanisms may be actuated via mechanical and/or electromechanical mechanisms. Accordingly, a brake mechanism may be actuated by the controller via a control signal or may be actuated directly by the user via a mechanical lever. Further, a brake control signal or signals may be generated by a controller and/or may be generated as a result of a user input. The brake mechanisms may include frictional elements, locking pawls, viscous elements, etc.

A top view illustration of the handle shown in FIG. 4A is shown in FIG. 4B. The control knobs 421, 423 may be coupled to the force transmitting members which may include dual racks. For example, the force transmitting member which is coupled to the control knob 423 and/or actuator 408-1 may include racks 409-1A and 409-1B. Further, the force transmitting member which is coupled to the control knob 421 and/or actuator 408-2 may include racks 409-2A and 409-2B.

A flow chart illustrating a process according to the present invention is shown in FIG. 5. Process 500 may be performed using one more computers, e.g. the processor 132 of the control unit 130, communicating over a network such as, for example, a LAN (local-area network), a WAN (wide-area network), the Internet, etc. The process 500 can include one of more of the following steps, acts and/or operations. Further, one or more of these operations may be combined and/or separated into sub-operations, if desired.

With reference to FIG. 5, in step/act/operation 502, the process controls one or more of the TEE sensor arrays to acquire image information relating to a current location. The current location may correspond with location information obtained from one or more encoders and/or external location devices to determine location/orientation of one or more of the sensors arrays. The image information may include image information relating to a current image volume V. The process may then continue to act 504.

In act 504, the acquired image information may be processed to obtain desired information. For example, the processing may include digital signal processing so as to filter desired/undesired image information. Additionally, the image location may be stored with current location information for later use. The process may then continue to act 506.

In act 506, the process may determine whether the one or more of the sensor arrays is in a desired location/orientation. For example, the location/orientation may be determined by comparing image information obtained in act 502 and/or processed in act 504 with a table look up or other information such as, for example, a user's desired location/configuration, and/or the location of another device. If one or more of the sensor arrays are in a desired location, the process may repeat act 502. However, if one or more of the sensor arrays are determined not to be in a desired position and/or orientation, the process may continue to act 508.

In act 508, the process may calculate a desired position and/or orientation for one or more of the TEE sensor arrays. The desired position/orientation may correspond with a position/orientation input by a user, calculated by the system, and/or a position/orientation which corresponds with a current position of another device (e.g., an ablation catheter or a further endoscopic device for imaging) and/or a tissue volume of interest. Further, the system may include, for example, a menu selection to an enable a user to select between a vertical and/or horizontal position for the TEE 122 shown in FIG. 1 (e.g., see FIGS. 3A and 3B). Accordingly, the process may continually determine positions of one or more surgical devices and calculate a desired position for the TEE sensor array according to the present system.

The desired location may also be determined by calculating an incremental step Δᵢ (where i corresponds with a specific actuator of the one or more of the actuators) for one or more of the actuators. The incremental step Δᵢ may apply to an output of the i^{th} actuator. Further, the incremental step Δᵢ may apply to radial and/or linear movements of an actuator or parts thereof. Further, the process may refer to stored information such as, for example, a look-up table etc. stored in the memory 164 shown in FIG. 1, to determine a desired position and/or orientation for the one or more sensor arrays. After calculating a desired position and/or orientation for the one or more sensor arrays, the process may continue to act 510.

In act 510, the process controls one or more of the actuators in accordance with the desired location that was calculated in act 508. For example, with reference to FIGS. 1, 3A and 3B, the control unit 130 may obtain image information corresponding with the location and/or orientation of the sensor arrays shown in FIG. 3A and control one or more of the actuators 101-1, 101-2, 108-1, 108-2, and/or 177 so as to position and/or orient the sensor arrays to a final position as shown in FIG. 3B. Information received from the sensor array 222 and/or 227 may be used to, for example, determine the distance between a tip of the imaging endoscopic device 104 and a wall of a subject mass. Using the information received from one or more of the sensor arrays such as sensor arrays 222 and/or 227, the control unit 30 may control to prevent penetration of the mass. Further, the position and/or orientation of the sensor arrays can be controlled so as to correspond with the position and/or orientation of another surgical instrument such as, for example, a balloon or ablation catheter, etc. Accordingly, real time information may be obtained relating to the other surgical instrument. For example, a tracking function performed by the control unit 130 may obtain imaging information relating to another surgical instrument and control one or more of the actuators and/or TEE sensor arrays such that the position, orientation, and/or configuration of one or more of the TEE sensor arrays is in accord with the position of the other surgical instrument. Accordingly, the TEE sensor array may provide real-time image information relating to another surgical instrument during a surgical routine even as the location of the other surgical instrument is varied.

It is also envisioned that the control unit may also include an automatic retrieval function wherein one or more of the actuators are controlled so that the imaging system may be automatically removed from the subject mass. For example, retrieval may be activated by setting all the actuator voltage to zero, except the actuator(s) that performs the removal or retrieval, depending on the application, where more than one actuator may be used concurrently and/or sequentially to effectuate the retrieval. Accordingly, upon selecting a retrieval mode, the control unit may control, for example, one or more of the actuators coupled to the flexible section such that flexible section is articulated and/or may control an actuator located in the telescopic assembly so that the imaging endoscopic device may be straightened and/or removed from the subject mass.

Further, the imaging system may initially provide various views such as, for example, front and/or side views for a user's convenience. The imaging system may also provide a modified C-scan image that is an image of a selected surface perpendicular to the front and side view planes over the scanned volume V. A user may manually select (or the system may automatically select) the surface to be shown in the modified C-scan image. The imaging system may also generate these and other orthographic projection views in real time, e.g., at a frame rate above 15 Hz (and preferably above 20 Hz, or in the range of about 30 Hz to 100 Hz).

The ultrasound imaging system may include shielding so that it is shielded from receiving/transmitting unwanted electromagnetic (EM) and/or radio frequency (RF) radiation. Accordingly, the shielding may include any suitable shielding which may prevent the transmission/reception of unwanted EM and/or RF fields. Accordingly, the ultrasound imaging system may include adequate shielding for use in surgical environments such that it may be used in proximity with electro-surgical units (ESUs) which may generate broad spectrum electromagnetic energy. Accordingly, the shielding may include shielding as set forth in U.S. Pat. No. 6,776,758 and U.S. Patent Publication No. 2004/0073118 each of which is incorporated herein as if set out in its entirety.

Further, although two control cables 134, 174 are shown, these cables may be combined so as to form a single cable and/or may be transmitted via, for example, a wired or wireless link. Further, the cables 134, 174, or portions thereof as well as any other connections, may include a wireless link. Further, one or more components of the ultrasound imaging system 100, may be located in a remote location. For example, the control unit 130, and/or parts thereof, may be located at a remote location from the imaging endoscopic device 104 and communicate via a wired and/or wireless link.

Certain additional advantages and features of this invention may be apparent to those skilled in the art upon studying the disclosure, or may be experienced by persons employing the novel system and method of the present invention, chief of which is that a more reliable and easily maneuvered ultrasound imaging apparatus and method which may be remotely operated is provided. Another advantage of the present systems and devices is that conventional ultrasound imaging devices can be easily upgraded to incorporate the features and advantages of the present systems and devices.

Of course, it is to be appreciated that any one of the above embodiments or processes may be combined with one or more other embodiments and/or processes or be separated and/or performed amongst separate devices or device portions in accordance with the present systems, devices and methods.

It is further envisioned that the probe according to the present system may be used with other types of endocavity probes. For example, the endoscopic devices for imaging according to the present system may include various device types such as TEE, transnasal, transvaginal, transrectal, endco-cavity (e.g., a transducer with a shaft at the end with ultrasound array that moves the array to touch or come close to a mass that is to be operated on for surgical application, for example, inserted through a natural opening or an opening made by a surgeon. The endoscopic devices for imaging according to the present system may be manually and/or automatically controlled, including manual/automatically control from a remote location, i.e., remote from the location of the procedure, where the controller and associated devices such as display, I/O device, memory, are operationally connected to a local controller or processor, through a network, such as the Internet. Control and others signals including image signals may be transmitted and/or received through any means, wired or wireless, for example.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

In interpreting the appended claims, it should be understood that:

a) the word “comprising” does not exclude the presence of other elements or acts than those listed in a given claim;

b) the word “a” or “an” preceding an element does not exclude the presence of a plurality of such elements;

c) any reference signs in the claims do not limit their scope;

d) several “means” may be represented by the same item or hardware or software implemented structure or function;

e) any of the disclosed elements may be comprised of hardware portions (e.g., including discrete and integrated electronic circuitry), software portions (e.g., computer programming), and any combination thereof;

f) hardware portions may be comprised of one or both of analog and digital portions;

g) any of the disclosed devices or portions thereof may be combined together or separated into further portions unless specifically stated otherwise;

h) no specific sequence of acts or steps is intended to be required unless specifically indicated; and

i) the term “plurality of” an element includes two or more of the claimed element, and does not imply any particular range of number of elements; that is, a plurality of elements may be as few as two elements, and may include an immeasurable number of elements.

What is claimed is:

1. An ultrasound imaging probe, comprising:
a body portion comprising a rigid section defining at least part of a cavity;
a first electromechanical actuator located in the body portion;
a second electromechanical actuator located in the body portion;
a flexible coupled to the body portion, the flexible portion comprising a plurality of articulating elements;
a distal part coupled to the flexible portion and defining at least another part of the cavity;
an ultrasonic sensor array situated in the distal part;
a controller for providing control signals;
a first force transmitting member coupled to the first electromechanical actuator and at least one of the plurality of articulating elements so as to transfer a force from the first electromechanical actuator to at least one of the articulating elements; and
a second force transmitting member coupled to the second electromechanical actuator and at least another of the plurality of articulating elements so as to transfer a force from the second electromechanical actuator to the other articulating element of the plurality of articulating elements in response to the control signals from the controller.

2. The ultrasonic imaging probe of claim 1, wherein the controller is configured to electronically steer a beam from the ultrasonic sensor array in response to a manual manipulation of a joystick by a user to provide volumetric imaging in three dimensions.

3. The ultrasound imaging probe of claim 1, further comprising one or more control knobs suitable for grasping by a user, the one or more control knobs being attached to the body portion and coupled to the first or second force transmitting members.

4. The ultrasonic imaging probe of claim 1, wherein the first and the second force transmitting members comprise a geared rack and a cable.

5. The ultrasonic imaging probe of claim 1, further comprising a third actuator coupled to the distal part and which rotates the ultrasonic sensor array about a longitudinal axis of the distal part.

6. The ultrasonic imaging probe of claim 5, further comprising a fourth actuator coupled to the body portion and which rotates the body portion about a longitudinal axis of the body portion.

7. The ultrasonic imaging probe of claim 1, further comprising a telescoping assembly coupled to the body portion and which can linearly displace the body portion a predetermined distance.

8. The ultrasonic imaging probe of claim 1, further comprising at least one encoder coupled to the first or second electromechanical actuators and which provides articulation information corresponding to an articulation of the flexible portion.

9. A method for controlling an imaging probe using a controller, the method comprising the acts of:
driving, by the controller, an ultrasonic array mounted in a cavity of a distal portion of the imaging probe;
receiving, by the controller, image information from the ultrasonic array;
activating, by the controller, one or more electromechanical located in at least part of the a body portion situated opposite the distal portion; and
articulating, by the one or more electromechanical actuators, a flexible portion situated between the body portion and the distal portion, the flexible portion comprising a plurality of articulating elements.

10. The method for controlling an ultrasound imaging probe of claim 9, wherein the articulating act further comprises:
rotating, by a user, one or more control knobs that are attached to the body portion and coupled to corresponding force transmitting members; and
transmitting a force from at least one of the control knobs to at least one of the articulating elements.

11. The method for controlling an ultrasound imaging probe of claim 9, further comprising rotating the sensor array about a longitudinal axis of the distal part using an actuator which is coupled to the distal portion and the flexible portion.

12. The method for controlling an ultrasound imaging probe of claim 9, further comprising locking the distal portion in a desired location using a brake mechanism controlled by the controller.

13. The method for controlling the ultrasound imaging probe of claim 9, further comprising displacing the body portion a predetermined linear distance using a telescoping assembly coupled to the body portion and controlled by the controller.

14. The method for controlling the ultrasound imaging probe of claim 9, further comprising:
transmitting, from one or more encoders, articulation information to the controller; and
determining, by the controller, a position or orientation of the distal portion using the articulation information.

15. An ultrasound imaging system, comprising:
a controller which receives image information;
an input device coupled to the controller and arranged to receive an input from a user;
a display coupled to the controller and which displays information corresponding to the image information received by the controller; and
a probe comprising:
a body portion comprising a rigid section defining at least part of a cavity;
a first electromechanical actuator located in the body portion;
a second electromechanical actuator located in the body portion;
a flexible portion located coupled to the body portion, the flexible portion comprising a plurality of articulating elements;
a distal part coupled to the flexible portion and defining at least part of the cavity;
an ultrasonic sensor array situated in the distal part and which transmits image information to the controller;
a first force transmitting coupled to the first electromechanical actuator and at least one of the plurality of articulating elements so as to transfer a force from the first electromechanical actuator to at least one of the articulating elements; and
a second force transmitting member coupled to the second electromechanical actuator and at least another of the plurality of articulating elements so as to transfer a force from the first electromechanical actuator to the other of the plurality of articulating elements.

16. The ultrasound imaging system of claim 15, further comprising one or more control knobs suitable for grasping by a user, the one or more control knobs being attached to the body portion and coupled to the first or second force transmitting members.

17. The ultrasonic imaging system of claim 15, wherein the first and second force transmitting members comprise a geared rack and a cable.

18. The ultrasonic imaging system of claim 15, further comprising a third actuator coupled to the distal part and which rotates the ultrasonic sensor array about a longitudinal axis of the distal part.

19. The ultrasonic imaging system of claim 15, further comprising a fourth actuator coupled to the body portion and which rotates the body portion about a longitudinal axis of the body portion.

20. The ultrasonic imaging system of claim 15, further comprising a telescoping assembly coupled to the body portion and which can linearly displace the body portion a predetermined distance.
